(19) United States
(12) Patent Application Publication
(10) Pub. No.: US 2020/0147451 A1
(43) Pub. Date: May 14, 2020

(54) METHODS, SYSTEMS, AND NON-TRANSITORY COMPUTER READABLE MEDIA FOR ASSESSING LOWER EXTREMITY MOVEMENT QUALITY

(71) Applicants: The University of North Carolina at Chapel Hill, Chapel Hill, NC (US); Duke University, Durham, NC (US)

(72) Inventors: Barnett Samuel Frank, Chapel Hill, NC (US); Darin Anthony Padua, Durham, NC (US); Michael Joseph Mazzoleni, Morrisville, NC (US); Brian Patrick Mann, Durham, NC (US)

(21) Appl. No.: 16/632,054

(22) PCT Filed: Jul. 17, 2018
(86) PCT No.: PCT/US2018/042451

§ 371 Date: Jan. 17, 2020

Related U.S. Application Data
(60) Provisional application No. 62/533,523, filed on Jul. 17, 2017.

(51) Int. Cl. A63B 24/00; A61B 5/00; A61B 5/11; G06N 20/10; G06K 9/00; G06K 9/62

(52) U.S. Cl. CPC A63B 24/0062; A61B 5/7275; A61B 5/1123; A61B 2562/0219; G06K 9/00342; G06K 9/6267; G06N 20/10

(57) ABSTRACT

A system for assessing lower extremity movement quality includes one or more user sensors, at least one processor, and a movement evaluator implemented using the at least one processor. The movement evaluator is configured, by virtue of appropriate programming, for receiving movement data from the one or more user sensors during a user's performance of a movement task; extracting one or more movement features from the movement data, each movement feature characterizing a respective aspect of a movement pattern of the user's performance that is associated with lower extremity injury risk; and classifying the movement pattern into a classified risk category for lower extremity injury for the user based on the one or more movement features.

### PRIORITY CLAIM

This application claims the benefit of U.S. Provisional Patent Application Ser. No. 62/533,523 filed Jul. 17, 2017, the disclosure of which is incorporated herein by reference in its entirety.

### TECHNICAL FIELD

This specification relates generally to assessing lower extremity movement quality, e.g., by applying machine learning algorithms to movement data from user sensors to classify movement patterns that are associated with lower extremity injury risk.

### BACKGROUND

Clinical assessment tools can provide a simple and efficient method for identifying problems in the musculoskeletal system that may result in athletic injury. Sports medicine clinicians can often use the results of such assessment tools to develop effective intervention programs for injury prevention and rehabilitation (Padua et al., 2009, 2011, 2015). Many individuals are at risk to develop anterior cruciate ligament (ACL) or other serious lower extremity injuries, and those who experience such injuries often struggle to return to their normal active lifestyle and suffer from pain and disability (Noyes et al., 1983a,b; Brophy et al., 2012; Moksnes et al., 2013). Effective screening methods such as the Landing Error Scoring System (LESS) have been developed to identify movement patterns during a jump-landing task that are related to lower extremity injury risk (Padua et al., 2009, 2011, 2015). Abnormal lower extremity movement patterns are modifiable, so identifying injury risk patterns prior to injury can result in effective interventions. However, screening methods such as the LESS are reliant on subjective assessments from trained professionals, which limits the scope and scale of their applicability.

Accordingly, there exists a need for an objective and autonomous system to classify lower extremity movement patterns, and for methods and systems for enabling assessments to take place in a field testing environment as opposed to in a controlled laboratory setting.

### SUMMARY

A system for assessing lower extremity movement quality includes one or more user sensors, at least one processor, and a movement evaluator implemented using the at least one processor. The movement evaluator is configured, by virtue of appropriate programming, for receiving movement data from the one or more user sensors during a user's performance of a movement task; extracting one or more movement features from the movement data, each movement feature characterizing a respective aspect of a movement pattern of the user's performance that is associated with lower extremity injury risk; and classifying the movement pattern into a classified risk category for lower extremity injury for the user based on the one or more movement features.

The subject matter described in this specification may be implemented in hardware, software, firmware, or combinations of hardware, software and/or firmware. In some examples, the subject matter described in this specification may be implemented using a non-transitory computer-readable medium storing computer executable instructions that when executed by one or more processors of a computer cause the computer to perform operations.

Computer-readable media suitable for implementing the subject matter described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer-readable medium that implements the subject matter described in this specification may be located on a single device or computing platform or may be distributed across multiple devices or computing platforms.

### BRIEF DESCRIPTION OF THE DRAWINGS

FIG. 1 illustrates an example of a participant performing a jump-landing task;

FIG. 2 illustrates visual examples of excellent and poor lower extremity movement patterns;

FIGS. 3A-B illustrate examples of the raw data collected from the accelerometer and force plates during a jump-landing trial;

FIGS. 4A-B show ROC curves derived from the accelerometer-based SVM and force plate-based SVM;

FIGS. 5A-B illustrate example systems for assessing lower extremity movement quality;

FIGS. 6A-C illustrate example movement data from a countermovement jump, a medicine ball throw, and an agility test;

FIGS. 7A-B illustrate example movement data from a chest-mounted tri-axial accelerometer and from a chest-mounted tri-axial gyroscope during treadmill running; and

FIG. 8 is a flowchart of an example method for assessing lower extremity movement quality.

### DESCRIPTION

This specification describes methods and systems for assessing lower extremity movement quality, e.g., by applying machine learning algorithms to movement data from user sensors to classify a movement pattern that is associated with lower extremity injury risk.

For purposes of illustration, this specification describes a study performed to develop an accurate method to classify lower extremity movement patterns during a jump-landing task using a support vector machine (SVM). The methods and systems for assessing lower extremity movement quality, however, may be used in various other appropriate settings than those described in the study and may be implemented using any appropriate technology.

In the study, forty female athletes each performed three trials of a jump-landing task. Two digital video cameras were used to record the frontal and sagittal perspectives during the jump-landing tasks. The video data was evaluated to classify the lower extremity movement patterns of the participants as either excellent (n=20) or poor (n=20) using the Landing Error Scoring System (LESS) assessment method. Data was also collected from two force plates and a chest-mounted single-axis accelerometer. Fourteen features were extracted from the force plate data and four features were extracted from the accelerometer data. A principal component analysis (PCA) was applied to the force plate data to reduce the dimensionality of the feature vector. Five principal components (PC's) were retained, explaining 94.4% of the variance in the force plate data.

Two separate linear SVM classifiers were trained using the accelerometer data and the force plate data, respectively, with the LESS assessment providing the classification labels during training and evaluation. The accelerometer-based SVM had an overall classification accuracy of 80.0%, and the force plate-based SVM had an overall classification accuracy of 87.5%. These findings suggest that a linear SVM can successfully classify lower extremity movement patterns during a jump-landing task using force plate and accelerometer data, with force plate data providing greater classification accuracy.

1. Introduction

Clinical assessment tools can provide a simple and efficient method for identifying problems in the musculoskeletal system that may result in athletic injury. Sports medicine clinicians can often use the results of such assessment tools to develop effective intervention programs for injury prevention and rehabilitation (Padua et al., 2009, 2011, 2015). Many individuals are at risk to develop anterior cruciate ligament (ACL) or other serious lower extremity injuries, and those who experience such injuries often struggle to return to their normal active lifestyle and suffer from pain and disability (Noyes et al., 1983a,b; Brophy et al., 2012; Moksnes et al., 2013). Effective screening methods such as the Landing Error Scoring System (LESS) have been developed to identify movement patterns during a jump-landing task that are related to lower extremity injury risk (Padua et al., 2009, 2011, 2015). Abnormal lower extremity movement patterns are modifiable, so identifying injury risk patterns prior to injury can result in effective interventions. However, screening methods such as the LESS are reliant on subjective assessments from trained professionals, which limits the scope and scale of their applicability. The development of an objective and autonomous system to classify lower extremity movement patterns during a jump-landing task would provide a powerful and objective clinical assessment tool. Integrating such a system with portable and inexpensive sensors would provide further benefits by enabling assessments to take place in a field testing environment as opposed to in a controlled laboratory setting.

Bittencourt et al. (2016) recently advocated for a paradigm shift in the field of sports injury prevention, encouraging researchers to move away from a reductionist view of injury factor identification to a more complex systems approach of injury pattern recognition. One method for accomplishing this objective is through the application of machine learning algorithms, which have the ability to recognize complex patterns and create data-driven prediction models (Shalev-Shwartz and Ben-David, 2014). Machine learning techniques have been applied to a wide assortment of biomechanical studies including gait recognition (Jonic et al., 1999; Begg and Kamruzzaman, 2005; Clermont et al., 2017), runner classification (Maurer et al., 2012; Kobsar et al., 2014), patient ailments and treatments (Silver et al., 2006; Deluzio and Astephen, 2007; Muniz et al., 2010; Labbe et al., 2011), and fall detection (Maki, 1997; Lutrek and Kalua, 2009; Phinyomarksz et al., 2015). Such classification methods provide an objective and analytical alternative to methods that have previously relied on subjective criteria.

A commonly used machine learning algorithm is the support vector machine (SVM), which constructs a hyperplane to maximize the margin of separation between a classified data set (Boser et al., 1992; Cortes and Vapnik, 1995). Begg and Kamruzzaman (2005) used an SVM to analyze how kinematic and kinetic data extracted from the gait of young and elderly adults could be used to classify gait patterns. Fukuchi et al. (2011) used an SVM to detect age-related changes in running kinematics. Chan et al. (2010) used an SVM to classify ankle sprains. Wu and Wang (2008) implemented a principal component analysis (PCA) for feature vector dimensionality reduction and utilized an SVM with nonlinear kernels (polynomial and radial basis function) to classify gait patterns. Other supervised learning approaches such as information gain and artificial neural networks (ANN's) have been used to examine the effects of midsole resilience and upper shoe structure on running kinematics (Onodera et al., 2017). Muniz et al. (2010) compared the performance of logistic regression (LR), ANN, and SVM to classify the gaits of subjects diagnosed with Parkinson's disease. Bennetts et al. (2013) applied k-means clustering to study peak plantar pressure distributions for diabetic patients. Kobsar et al. (2014) used tri-axial accelerometer data and a discriminant analysis to classify runners based on their experience level and training background. However, to date, we are unaware of any study that has applied machine learning algorithms to classify lower extremity movement patterns during a jump-landing task that are associated with increased lower extremity injury risk.

The purpose of this study was to develop an accurate and autonomous method for classifying lower extremity movement patterns during a jump-landing task using a linear SVM. Video analysis was implemented to classify the participants' lower extremity movement patterns using the LESS assessment method. Data was collected from two force plates and a chest-mounted single-axis accelerometer. Two separate linear SVM classifiers were trained using the accelerometer data and the force plate data, respectively, with the LESS assessment providing the classification labels during training and evaluation.

2. Methods

2.1. Participants

Forty female athletes were recruited for this study. The participants were all physically active, participated in field or court sports, had no history of lower extremity injuries, and were between the ages of 18-25. The participants were evaluated while performing a jump-landing task using the LESS assessment method (Padua et al., 2009, 2011, 2015), with one group exhibiting excellent lower extremity movement patterns (n=20; age: 20.6±1.9 yr; mass: 64.5±7.8 kg; height: 1.67±0.07 m) and the other group exhibiting poor lower extremity movement patterns (n=20; age: 20.4±1.3 yr; mass: 60.9±6.1 kg; height: 1.69±0.07 m). Informed written consent was obtained from each participant, and the testing protocol was approved by the institutional review board (IRB #14-3298).

2.2. Data Collection

Each participant performed three trials of a jump-landing task from a 30 cm platform placed at a distance of one-half their body height from a landing target consisting of two force plates located in front of the jump platform. The participants were instructed to jump forward so that both feet left the platform simultaneously, to land in the middle of the force plates with one foot on each force plate, and to jump for maximal height immediately after landing.

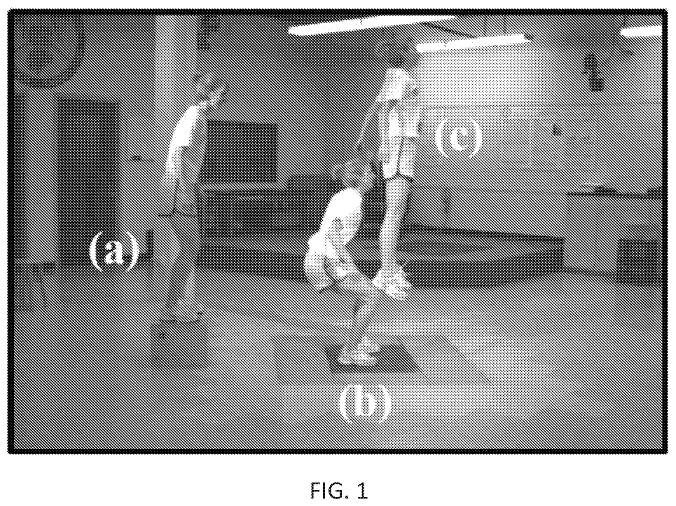

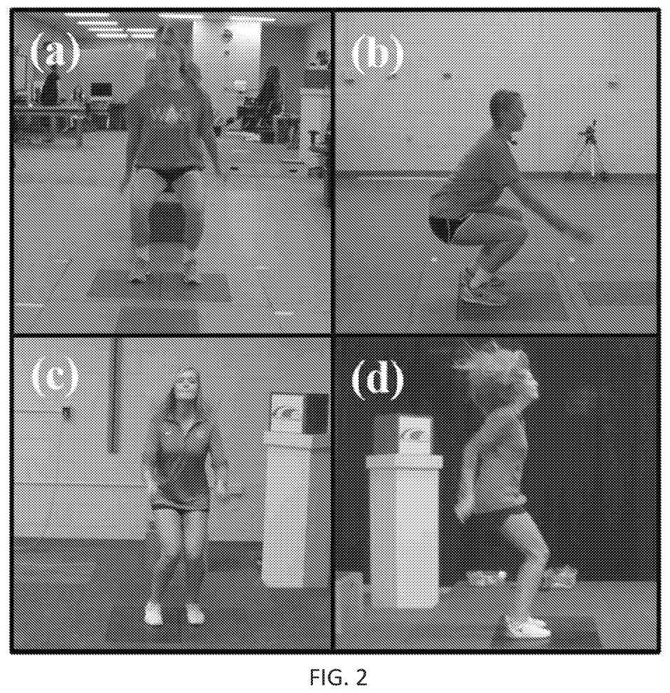

FIG. 1 illustrates an example of a participant performing the jump-landing task by: (a) jumping down from the platform; (b) landing on the force plates; (c) immediately jumping vertically as high as possible.

The participants were allowed to practice until they felt comfortable with the jump-landing task and were able to complete it correctly. Mistrials were excluded and repeated if the participant did not perform the jump-landing correctly. Two digital video cameras (HERO3+; GoPro, Inc.; San Mateo, Calif.) were positioned 3 m away from the participant to record the frontal and sagittal perspectives during the jump-landing tasks. Two expert raters evaluated the videos using free computer software (QuickTime; Apple, Inc.; Cupertino, Calif.) and classified each participant as exhibiting either excellent or poor lower extremity movement patterns during the jump-landing task using the LESS assessment method (Padua et al., 2009, 2011, 2015). If the two evaluators disagreed on the classification assessment, then a third evaluator was consulted.

FIG. 2 illustrates visual examples of excellent and poor lower extremity movement patterns. In FIG. 2, a participant demonstrates excellent lower extremity movement patterns by: (a) exhibiting no medial knee displacement in the frontal plane at maximum knee flexion; (b) exhibiting a “soft” landing with large displacement of the trunk, hips, and knees in the sagittal plane. A participant demonstrates poor lower extremity movement patterns by: (c) exhibiting medial knee displacement in the frontal plane at maximum knee flexion; (d) exhibiting a “stiff” landing with very little displacement of the trunk, hips, and knees in the sagittal plane.

Data was collected from the two force plates (FP4060-10; Bertec Corp.; Columbus, Ohio) and a chest-mounted inertial measurement unit (Armour39; Under Armour, Inc.; Baltimore, Md.). The force plates collected tri-axial ground reaction force and moment data for each foot, but only the vertical ground reaction force (VGRF) data was used in this study. The inertial measurement unit contained a tri-axial accelerometer, tri-axial gyroscope, and tri-axial magnetometer, but only the data collected from the accelerometer axis aligned with the vertical orientation of the participant was used in this study. The force plate data was sampled at 1000 Hz and the accelerometer data was sampled at 200 Hz. A zero-phase second-order high-pass Butterworth filter with a cutoff frequency of 0.2 Hz was applied to the accelerometer data to remove the DC offset. All aspects of post-processing, data analysis, and statistical analysis were carried out using MATLAB software (MATLAB R2016a; MathWorks, Inc.; Natick, Mass.).

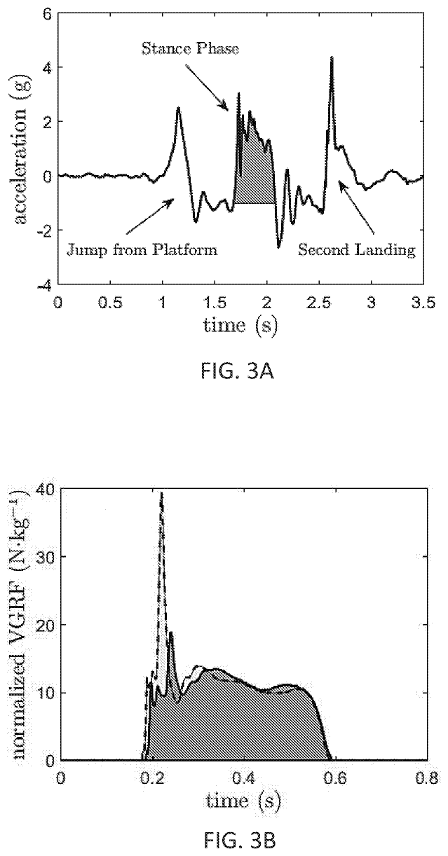

Examples of the raw data collected from the accelerometer and force plates during a jump-landing trial can be seen in FIGS. 3A-B. FIGS. 3A-B illustrate examples of the raw data collected from the (FIG. 3A) accelerometer and (FIG. 3B) force plates during a jump-landing trial. The shaded regions on the plots represent the stance phase. In FIG. 3A, labels are provided to distinguish between the three main portions of the jump-landing task. In FIG. 3B, the left force plate data is represented by the solid line and the right force plate data is represented by the dashed line. These data sets came from a participant who exhibited poor lower extremity movement patterns.

2.3. Feature Extraction

The features used in the data analysis were calculated from the stance phase of the jump-landing task, as this is a critical portion of the test for classifying lower extremity movement patterns (Padua et al., 2009, 2011, 2015). The stance phase occurred when the participant landed on the force plates following the horizontal jump from the platform and began transitioning into the subsequent vertical jump. The stance phase was formally defined to begin when the first foot landed on the ground and to end when the last foot left the ground. Four features were calculated from the stance phase of the accelerometer data: ground contact time (TIME), pseudo-impulse during the first half (IMP₁), pseudo-impulse during the second half (IMP₂), and peak acceleration (PEAK). These features were selected to capture the timing and loading characteristics of the stance phase. The pseudo-impulse variables were defined as the area under the curve of the acceleration profile over the desired time interval (with a baseline value of −1 g). For reference, the stance phase and pseudo-impulse calculated from the accelerometer data are depicted in FIG. 3A.

Fourteen features were calculated from the stance phase of the force plate data: ground contact time (TIME), maximum impulse during the first half (IMP_{Max1}), total impulse during the first half (IMP_{Tot1}), maximum impulse during the second half (IMP_{Max2}), total impulse during the second half (IMP_{Tot2}), maximum impulse over the entire time duration (IMP_{Max}), maximum peak VGRF (PEAK_{Max}), average peak VGRF (PEAK_{Avg}), minimum time to peak VGRF (PEAK_{MinTime}), average time to peak VGRF (PEAK_{AvgTime}), maximum slope to peak VGRF (PEAK_{MaxSlope}), absolute difference between the ground contact times of the two feet (TIME_{Δ}), absolute time difference between the landings of the two feet at the beginning of the stance phase (TIME_{Δ1}), and absolute time difference between the takeoffs of the two feet at the end of the stance phase (TIME_{Δ2}). These features were selected to capture the timing, loading, and asymmetrical characteristics of the stance phase.

All of the variables involving VGRF measurements were normalized by body mass (IMP_{Max1}, IMP_{Tot1}, IMP_{Max2}, IMP_{Tot2}, IMP_{Max}, PEAK_{Max}, PEAK_{Avg}, PEAK_{MaxSlope}). All of the variables involving maximum or minimum values were based on comparisons between the right and left force plates (IMP_{Max1}, IMP_{Max2}, IMP_{Max}, PEAK_{Max}, PEAK_{MinTime}, PEAK_{MaxSlope}). All of the variables involving total values were based on the sum of the right and left force plates (IMP_{Tot1}, IMP_{Tot2}). All of the variables involving average values were based on the average of the right and left force plates (PEAK_{Avg}, PEAK_{AvgTime}).

2.4. Principal Component Analysis (PCA)

The number of features contained in the accelerometer feature vector (n=4) was small relative to the sample size of participants (n=40), so those features could be used directly in the subsequent SVM analysis. However, the number of features contained in the force plate feature vector was relatively large (n=14), so a PCA was implemented to reduce the dimensionality of the force plate feature vector and thereby decrease the risk of overfitting. PCA is a statistical procedure that creates a set of orthogonal principal components (PC's) to describe the original set of data (Joliffe, 2002). Prior to implementing the PCA, all variable distributions were normalized to have a mean of 0 and a standard deviation of 1. After implementing the PCA, the system's eigenvalues (λ's) were analyzed to determine the appropriate number of PC's to retain, and a varimax rotation was then applied to the retained PC's to obtain a simple structure and increase interpretability (see Kobsar et al. (2014) for a similar application).

2.5. Support Vector Machine (SVM) Classification

Two separate linear SVM classifiers were trained, one using the accelerometer feature vectors and the other using the force plate feature vectors (after implementing the PCA). An SVM is a supervised machine learning algorithm that constructs a hyperplane to maximize the margin of separation between a classified data set (Boser et al., 1992; Cortes and Vapnik, 1995). The variable distributions contained in the feature vectors were normalized to have a mean of 0 and a standard deviation of 1 to avoid artificially exaggerating the importance of certain features, and 10-fold cross-validation was applied to the data sets to help prevent overfitting during the training process. Each individual jump-landing trial was used in the training process (n=120). However, for classification purposes, the SVM scores of the individual trials were averaged for each participant (n=40).

2.6. Statistical Analysis

Descriptive statistics (mean±SD) were calculated for all of the variables contained in the feature vectors, as well as for all of the participants' demographic variables (age, mass, and height). The differences between the feature vectors of the two subject groups were examined using p-values calculated from an unpaired two-tailed Student's t-test with statistical significance set at α=0.05. Cohen's d was calculated to evaluate the effect size of these differences (Cohen, 1988).

Receiver operating characteristic (ROC) curves were generated using the SVM scores from both the individual trials and the averaged trials, and the area under the curve (AUC) was calculated for both cases (individual and averaged trials) to facilitate comparisons. An optimal cutoff point was selected on the averaged ROC curves to determine overall classification accuracy (ACC), true positive rate (TPR), and false positive rate (FPR). 95% confidence intervals (CI's) were calculated for each AUC.

3. Results

A statistical comparison between the feature vectors of the two subject groups can be seen in Table 1.

TABLE 1

| | Excellent | Poor | | |
|---|---|---|---|---|
| | Biomechanics | Biomechanics | | |
| | (n = 20) | (n = 20) | p-value | Cohen's d |
| Accelerometer Data | | | | |
| TIME (s) | 0.63 ± 0.13 | 0.46 ± 0.09 | <0.01 | 1.52 |
| IMP₁ (g · s) | 0.58 ± 0.07 | 0.49 ± 0.07 | <0.01 | 1.34 |
| IMP₂ (g · s) | 0.53 ± 0.06 | 0.47 ± 0.05 | <0.01 | 1.01 |
| PEAK (g) | 4.47 ± 2.14 | 5.12 ± 2.27 | 0.36 | 0.29 |
| Force Plate Data | | | | |
| TIME (s) | 0.65 ± 0.12 | 0.46 ± 0.09 | <0.01 | 1.68 |
| IMP_{Max1} (N · s · kg⁻¹) | 3.31 ± 0.34 | 2.89 ± 0.29 | <0.01 | 1.34 |
| IMF_{Tot1} (N · s · kg⁻¹) | 6.27 ± 0.61 | 5.43 ± 0.53 | <0.01 | 1.46 |
| IMP_{Max2} (N · s · kg⁻¹) | 2.67 ± 0.34 | 2.19 ± 0.25 | <0.01 | 1.60 |
| IMP_{Tot2} (N · s · kg⁻¹) | 5.13 ± 0.68 | 4.16 ± 0.43 | <0.01 | 1.72 |
| IMP_{Max} (N · s · kg⁻¹) | 5.94 ± 0.63 | 5.03 ± 0.49 | <0.01 | 1.59 |
| PEAK_{Max} (N · kg⁻¹) | 28.29 ± 6.84 | 31.21 ± 7.04 | 0.19 | 0.42 |
| PEAK_{Avg} (N · kg⁻¹) | 25.85 ± 6.52 | 28.14 ± 6.21 | 0.26 | 0.36 |
| PEAK_{MinTime} (ms) | 29.77 ± 8.60 | 35.35 ± 10.40 | 0.07 | 0.58 |
| PEAK_{AvgTime} (ms) | 32.44 ± 7.25 | 38.67 ± 10.43 | 0.03 | 0.69 |
| PEAK_{MaxSlope} (N · kg⁻¹ · ms⁻¹) | 1.02 ± 0.47 | 1.02 ± 0.57 | 0.99 | 0.01 |
| TIME_{Δ} (ms) | 8.48 ± 7.14 | 9.65 ± 9.19 | 0.66 | 0.14 |
| TIME_{Δ1} (ms) | 5.30 ± 4.28 | 4.88 ± 3.69 | 0.74 | 0.11 |
| TIME_{Δ2} (ms) | 6.28 ± 5.81 | 7.65 ± 7.21 | 0.51 | 0.21 |

The feature vector variables can be sorted into tiers related to their effect size. In general, the TIME and IMP variables have very large effect sizes, the PEAK variables have moderate effect sizes (with the exception of PEAK_{MaxSlope}, which has a very small effect size), and the TIME_{Δ} variables have small effect sizes.

The PCA was applied to the force plate feature vectors, and 5 PC's were retained following an analysis of the system's eigenvalues (all PC's retained had a corresponding λ>1.0). These 5 PC's explained 94.4% of the variance in the force plate data. The list of PC's associated with the force plate data and their corresponding eigenvalues and variable loadings following varimax rotation can be seen in Table 2. Cross-loadings are not shown.

TABLE 2

| PC₁ (λ = 5.68) | PC₂ (λ = 2.63) | PC₃ (λ = 2.12) | PC₄ (λ = 1.73) | PC₅ (λ = 1.12) |
|---|---|---|---|---|
| IMP_{Max} (0.98) | PEAK_{MinTime} (−0.96) | PEAK_{Max} (0.92) | TIME_{Δ2} (0.96) | TIME_{Δ1} (0.97) |
| TIME (0.97) | PEAK_{AvgTime} (−0.94) | PEAK_{Avg} (0.90) | TIME_{Δ} (0.91) | |
| IMP_{Max1} (0.96) | PEAK_{MaxSlope} (0.79) | | | |
| IMP_{Tot1} (0.95) | | | | |
| IMP_{Max2} (0.94) | | | | |
| IMP_{Tot2} (0.94) | | | | |

A statistical comparison between the feature vectors containing the normalized PC's of the two subject groups can be seen in Table 3.

TABLE 3

| | Excellent | Poor | | |
|---|---|---|---|---|
| | Biomechanics | Biomechanics | | |
| | (n = 20) | (n = 20) | p-value | Cohen's d |
| PC₁ | 0.68 ± 0.87 | −0.62 ± 0.65 | <0.01 | 1.70 |
| PC₂ | 0.22 ± 0.87 | −0.20 ± 1.07 | 0.18 | 0.44 |
| PC₃ | −0.26 ± 0.92 | 0.23 ± 1.02 | 0.12 | 0.50 |
| PC₄ | −0.14 ± 0.85 | 0.12 ± 1.11 | 0.41 | 0.26 |
| PC₅ | 0.15 ± 1.03 | −0.14 ± 0.96 | 0.37 | 0.29 |

The PC's can be sorted into tiers related to their effect size, and these tiers are similar to the ones established prior to implementing the PCA. PC₁ is associated with the TIME and IMP variables and has a very large effect size, PC_{2,3} are associated with the PEAK variables and have moderate effect sizes, and PC_{4,5} are associated with the TIMEΔ variables and have small effect sizes.

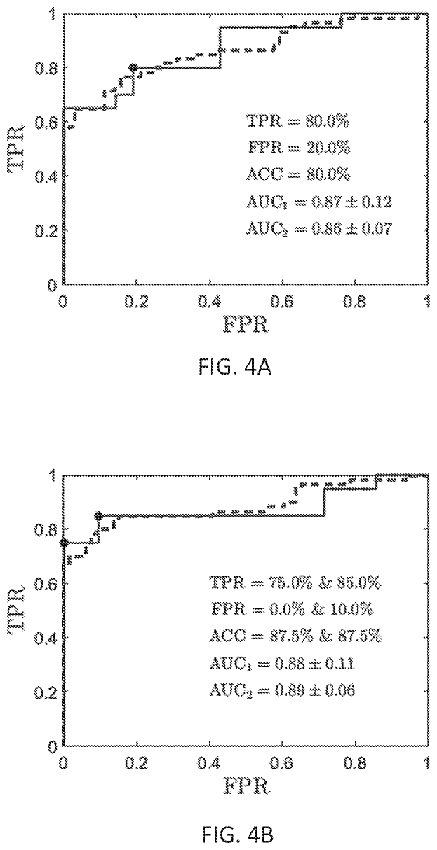

The SVM classification results are depicted in the ROC curves shown in FIGS. 4A-B. FIGS. 4A-B show ROC curves derived from the (FIG. 4A) accelerometer-based SVM and (FIG. 4B) force plate-based SVM. The solid lines represent the ROC curves generated using the SVM scores from the averaged trials (n=40) and the dashed lines represent the ROC curves generated using the SVM scores from the individual trials (n=120). The black circles represent the optimal cutoff points on the ROC curves. Note that the force plate-based SVM has two optimal cutoff points. The optimal true positive rates (TPR), false positive rates (FPR), and overall classification accuracies (ACC) are shown on the plots, along with the areas under the curve and their corresponding 95% CI's associated with the averaged trials (AUC1) and individual trials (AUC2).

The accelerometer-based SVM classifier had ACC=80.0%, TPR=80.0%, and FPR=20.0%. The AUC associated with the averaged trials was 0.87 (95% CI=0.75, 0.99) and the AUC associated with the individual trials was 0.86 (95% CI=0.79, 0.93). The force plate-based SVM classifier had two optimal cutoff points on its ROC curve, both with ACC=87.5%. The trade-off between the two cutoff points was related to their TPR and FPR, with one of the optimal cutoff points resulting in TPR=75.0% and FPR=0.0% and the other optimal cutoff point resulting in TPR=85.0% and FPR=10.0%. The AUC associated with the averaged trials was 0.88 (95% CI=0.77, 0.99) and the AUC associated with the individual trials was 0.89 (95% CI=0.83, 0.95).

4. Discussion

Both the force plate-based SVM and the accelerometer-based SVM were able to successfully classify lower extremity movement patterns with a high level of accuracy. Therefore, both methods have the potential to provide value as objective and autonomous clinical assessment tools. The classification accuracy of the force plate-based SVM (ACC=87.5%) was greater than the classification accuracy of the accelerometer-based SVM (ACC=80.0%). However, this is not surprising, since the force plates collected data directly at the location of impact during the jump-landing task, while the accelerometer collected data that was an indirect and dissipated representation of the impact event. Additionally, the force plate data was collected from two sensors as opposed to the accelerometer data being collected from one, and the force plate data was sampled at a higher frequency than the accelerometer data. However, the accuracy of the accelerometer-based SVM was still very close to the accuracy achieved by the force plate-based SVM. This is impressive, especially when considering the orders of magnitude price difference between force plates and accelerometers. Additionally, a force plate-based assessment method would typically be limited to applications in a laboratory setting, while a wearable accelerometer-based assessment method could be implemented at a much wider scale in a field testing environment.

The force plate-based SVM has two optimal cutoff points on its ROC curve, as shown in FIG. 4B, which indicates that a judgment call must be made if this algorithm were to be implemented in practice. Most researchers agree that lower extremity injury prevention programs pose no risk to participants, while ACL and other serious lower extremity injuries have devastating consequences (Padua et al., 2015). Therefore, it is recommended that a cutoff point should be selected to prioritize minimizing the potential for false positive predictions. For this case, the force plate-based SVM classifier would have FPR=0.0% and TPR=75.0%, indicating that 100% of the participants who exhibited poor lower extremity movement patterns would be correctly classified at the cost of incorrectly classifying 25% of the participants who exhibited excellent lower extremity movement patterns. Again, since lower extremity injury prevention programs are not harmful to the participants, this seems like an acceptable trade-off.

A traditional analysis of the variables contained in the feature vectors of the two subject groups might suggest that only the TIME, IMP, and PEAK_{AvgTime} variables have value for classifying lower extremity movement patterns, as these are the only variables contained in the feature vectors with significant differences between the two subject groups. However, it is important to consider that looking at the significant differences and effect sizes of individual variables is a reductionist approach that fails to account for complex patterns and interactions between variables. The SVM methods that were leveraged in this study searched the data for these patterns and variable relationships and generated sophisticated predictive models with excellent accuracy.

Machine learning algorithms have been applied to a large variety of biomechanical studies in recent years. However, to date, we believe that this is the first study that has applied machine learning algorithms to classify movement patterns during a jump-landing task that are associated with lower extremity injury risk. We also believe that the systems and methods employed in our study could be adapted in future studies to classify different movement patterns related to other injury risks.

One limitation of this study is the relatively small sample size (n=40) and narrow demographics of the subject pool, as all of the participants were young female athletes. Further validations with a larger sample size and broader demographics could be applied before applying this SVM classification approach for clinical assessment purposes. An additional limitation of this study was the fact that only VGRF data was analyzed from the force plates, even though six degrees of freedom (tri-axial ground reaction forces and moments) were available. Similarly, only the data collected from the accelerometer axis aligned with the vertical orientation of the participant was used in this study, even though the chest-mounted inertial measurement unit captured nine degrees of freedom (tri-axial accelerometer, tri-axial gyroscope, and tri-axial magnetometer). The decision to use only the VGRF and single-axis acceleration data was due to the relatively small sample size of participants (n=40), as increasing the size of the feature vectors could have resulted in overfitting. However, additional degrees of freedom undoubtedly contain valuable information, and further studies with larger sample sizes could utilize the extra features to increase classification accuracy.

The results of this study demonstrate that a linear SVM can accurately classify movement patterns during a jump-landing task that are associated with lower extremity injury risk. The objective and autonomous nature of this screening methodology eliminates the subjective limitations associated with many current clinical assessment tools. Integrating such a system with portable and inexpensive sensors would provide further benefits by enabling assessments to take place in a field testing environment as opposed to in a controlled laboratory setting.

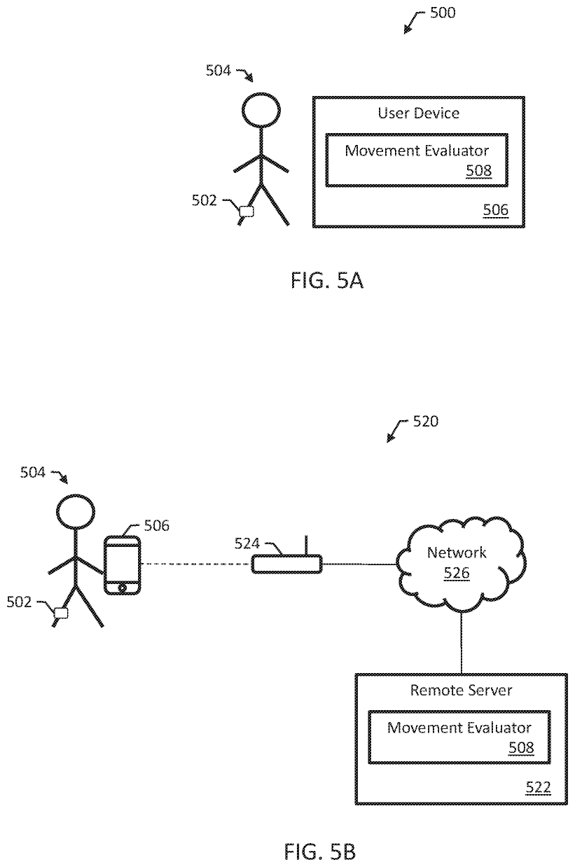

FIGS. 5A-B illustrate example systems for assessing lower extremity movement quality. FIG. 5A shows a first example system 500 including one or more user sensors 502 that may be worn by a user 504 or trained on the user 504 or otherwise situated to capture motion of the user 504. The system 500 includes a user device 506 and a movement evaluator 508 implemented on the user device 506. The user device 506 can be any appropriate computing device and typically includes at least one processor, memory storing instructions for the processor, and a display. The user device 506 may include a user input device and a wired or wireless communication system.

For example, the user device 506 may be a laptop, tablet, or mobile phone. The user sensors 502 or the user device 506 or both can be housed in a wearable fitness monitor. For example, the user sensors 502 may be embedded in a shoe or ankle bracelet or other user garment, and the user device 506 may be a smart watch. In some cases, some of the user sensors 502 are not wearable, e.g., where the user sensors 502 include a force plate situated on the ground for the user 504 to land on.

The movement evaluator 508 is programmed for receiving movement data from the user sensors 502 during the user's performance of a movement task and extracting one or more movement features from the movement data. Each movement feature characterizes a respective aspect of a movement pattern of the user's performance that is associated with lower extremity injury risk. The movement evaluator 508 is programmed for classifying the movement pattern into a classified risk category for lower extremity injury for the user based on the one or more movement features.

For example, classifying the movement pattern can include supplying the one or more movement features to a machine learning classifier trained using pre-classified training data for the movement task. The machine learning classifier can include a support vector machine (SVM) configured to construct a hyperplane to maximize, based on the pre-classified training data, a margin of separation between the risk categories. Extracting the one or more movement features can include performing a principle component analysis (PCA) to reduce a dimensionality of a feature set extracted from the movement data.

The movement evaluator 508 can be programmed to display, on a display of the user device 506, an indicator for the classified risk category for the user. For example, the movement evaluator 508 can display a text label for the classified risk category or a color representing the classified risk category.

FIG. 5B illustrates a second example system 520 including the user sensors 502 situated to capture motion of the user 504. The movement data is transmitted to a remote server 522 implementing the movement evaluator 508. The movement evaluator 508 on the remote server 522 is configured to classify movement patterns, e.g., for storage and analysis or to return classifications for local display.

For example, the user sensors 502 can transmit the movement data to the user device 506, which can then transmit the movement data to the remote server 522 (e.g., using a wireless router 524 and a data communications network 526 such as the Internet). In some examples, the user sensors 502 are configured with appropriate hardware for communicating directly with the wireless router 524 so that the user device 506 is optional. In some examples, the remote server 522 sends results from the classification to the user device 506 for display to the user 504.

In general, the movement task may be any appropriate task for assessing lower extremity motion quality, e.g., a jumping task; a running, jogging, or walking task; a cutting and sprinting task; a squatting task, a weight lifting task; a medicine ball toss task; or a combination of the listed tasks. For example, the following list illustrates optional examples of user sensors and movement features that can be used in various implementations for various movement tasks.

Jumping (Jump-Landing, Drop-Landing, Countermovement Jump)

1. Collect Data from Force Plates, IMU's, and/or Pressure Sensors

2. Extract Features
a. Takeoff Phase
   i. Peaks, Timing, Slopes, Areas under the Curve, Asymmetry
b. Flight Phase 1
   i. Timing, Stability, Asymmetry
c. Stance Phase 1
   i. Peaks, Timing, Slopes, Areas under the Curve, Asymmetry
d. Flight Phase 2
   i. Timing, Stability, Asymmetry
e. Stance Phase 2
   i. Peaks, Timing, Slopes, Areas under the Curve, Asymmetry

3. Run PCA or other similar feature reduction algorithm (optional)

4. Train Predictor Algorithm
a. Jump Performance Quality
b. Landing Performance Quality
c. Movement Quality
d. Balance Index

Running, Jogging, and/or Walking
1. Collect Data from Force Plates, IMU's, Pressure Sensors, GPS, RFID, HR monitors, VO2 monitors, and/or SmO2 monitors
2. Extract Features
   a. Foot Strike Features
      i. Pace, Cadence, Peaks, Ground Contact Time, Stride Length
   b. Time Series Features
      i. RMS, Speed-Normalized RMS, Step/Stride Regularity and Symmetry
3. Run PCA or other similar feature reduction algorithm (optional)
4. Train Predictor Algorithm
   a. Movement Quality
   b. Fatigue Index

Agility and Quickness (Cutting and Sprinting)

1. Collect Data from Force Plates, IMU's, and/or Pressure Sensors

2. Extract Features
a. Foot Strike Features
   i. Pace, Cadence, Peaks, Ground Contact Time, Stride Length
b. Time Series Features
   i. RMS, Speed-Normalized RMS, Step/Stride Regularity and Symmetry
c. Agility Features (during change of direction or stop/start)
   i. Peaks, Timing, Slopes, Areas under the Curve, Asymmetry

3. Run PCA or other similar feature reduction algorithm (optional)

4. Train Predictor Algorithm
a. Agility Index
b. Movement Quality

Squatting (Single-Leg and Double-Leg)

1. Collect Data from Force Plates, IMU's, and/or Pressure Sensors

2. Extract Features
a. Cadence, Peaks, Timing, Slopes, Areas under the Curve, Regularity, Asymmetry

3. Run PCA or other similar feature reduction algorithm (optional)

4. Train Predictor Algorithm
a. Movement Quality
b. Balance Index

Weight Lifting
1. Collect Data from Force Plates, IMU's, Pressure Sensors, HR monitors, VO2 monitors, and/or SmO2 monitors
2. Extract Features
   a. Cadence, Peaks, Timing, Slopes, Areas under the Curve, Regularity, Asymmetry
3. Run PCA or other similar feature reduction algorithm (optional)
4. Train Predictor Algorithm
   a. Performance Quality
   b. Movement Quality
   c. Fatigue Index
   d. Balance Index

Medicine Ball Toss

1. Collect Data from Force Plates, IMU's, and/or Pressure Sensors,

2. Extract Features
a. Peaks, Timing, Slopes, Areas under the Curve, Asymmetry

3. Run PCA or other similar feature reduction algorithm (optional)

4. Train Predictor Algorithm
a. Performance Quality
b. Movement Quality
c. Fatigue Index
d. Balance Index

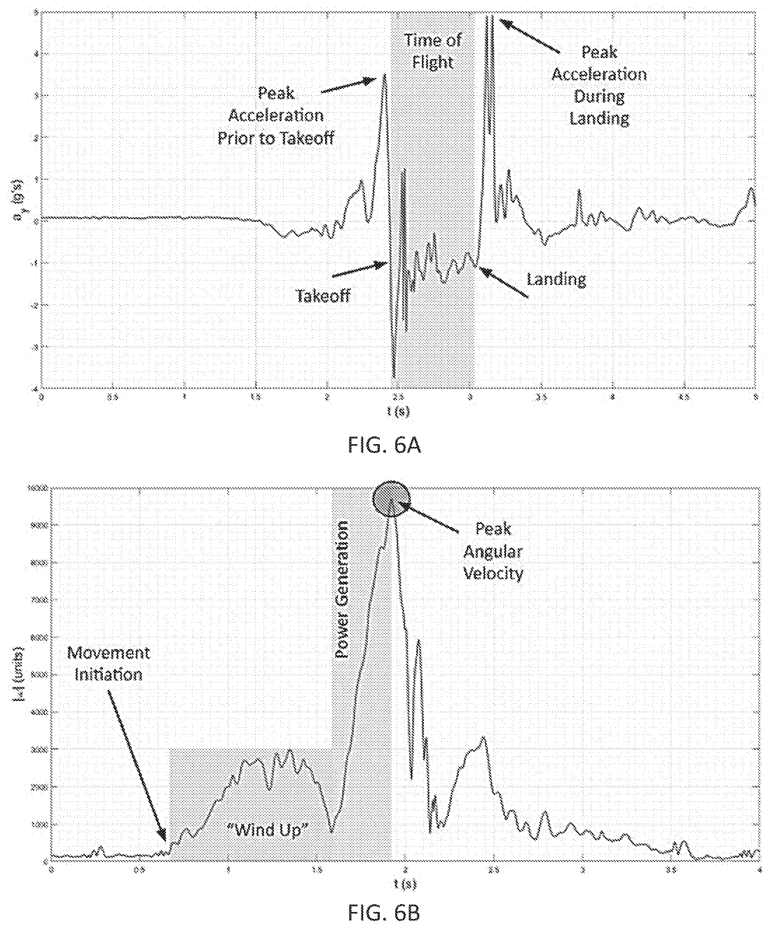

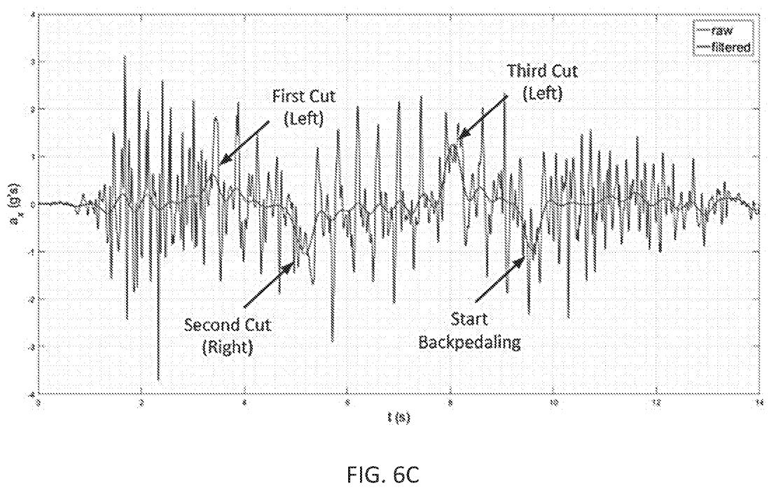

FIGS. 6A-C illustrate example movement data from a countermovement jump (FIG. 6A), a medicine ball throw (FIG. 6B), and an agility test (FIG. 6C).

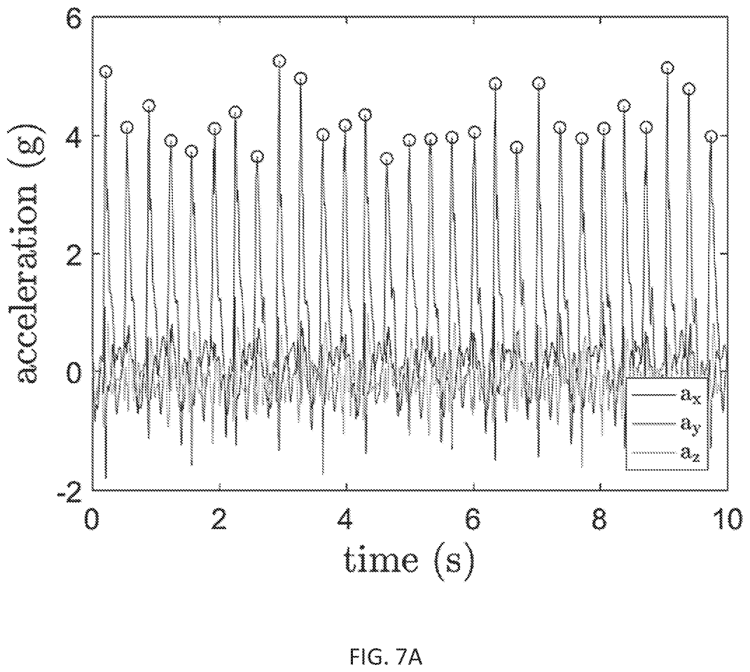

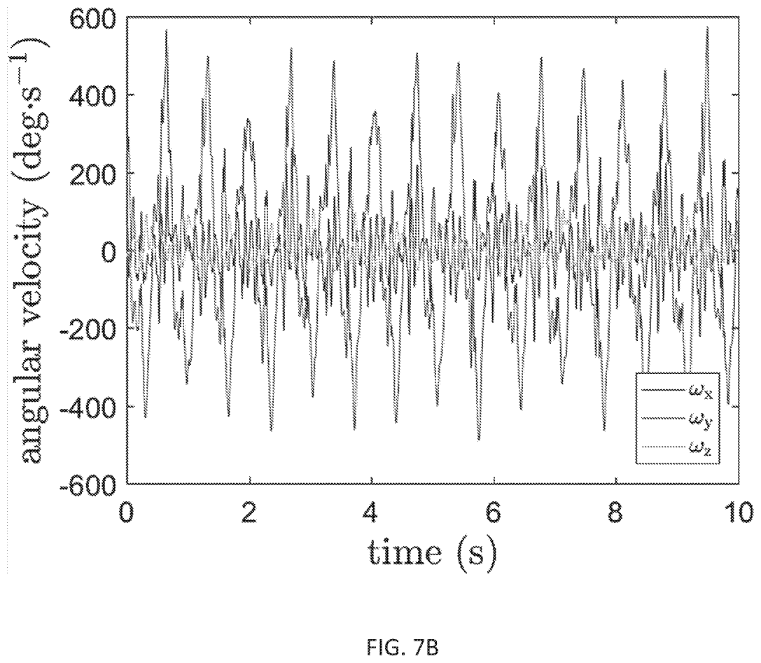

FIGS. 7A-B illustrate example movement data from a (FIG. 7A) chest-mounted tri-axial accelerometer and from a (FIG. 7B) chest-mounted tri-axial gyroscope during treadmill running. The following five features were calculated from the time series for all six degrees of freedom: root mean square (RMS), economy of acceleration/angular velocity (ECON), step regularity (REG1), stride regularity (REG2), and symmetry (SYM). The ECON variable was calculated by dividing RMS by the treadmill speed. The REG1 and REG2 variables were calculated using an unbiased autocorrelation procedure at a phase shift equal to the average step time and stride time, respectively. The SYM variable was calculated as the percent difference between REG1 and REG2. The average peak vertical acceleration and the coefficient of variation for the vertical acceleration were also calculated. These features were selected to capture the timing, loading, efficiency, regularity, and asymmetrical characteristics of the movement profile.

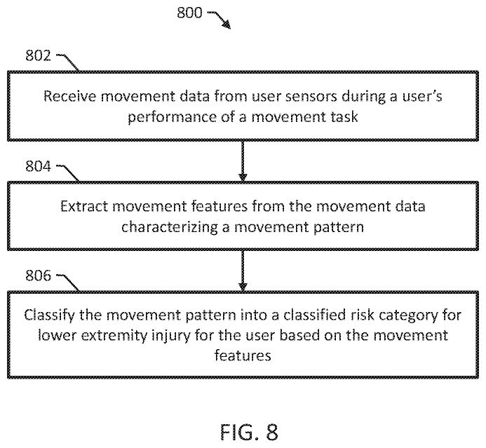

FIG. 8 is a flowchart of an example method 800 for assessing lower extremity movement quality. The method 800 can be performed by a movement evaluator implemented on a computer system.

The method 800 includes receiving movement data from one or more user sensors during a user's performance of a movement task (802). For example, receiving the movement data can include one or more of: receiving the movement data from one or more force plates during the user's performance of the movement task, receiving the movement data from one or more pressure sensors during the user's performance of the movement task, receiving the movement data from one or more video cameras during the user's performance of the movement task, and receiving the movement data from one or more wearable electronic fitness monitors worn by the user during the user's performance of the movement task.

A wearable electronic fitness monitor can include a combination of one or more accelerometers, gyroscopes, magnetometers, pressure sensors, GPS sensors, RFID sensors, HR monitors, VO2 monitors, and SmO2 monitors. The wearable electronic fitness device can be worn in any appropriate position on the user, for example, in clothing or apparel, in a chest strap, in a sports bra, in a waistband or belt, in footwear, in headphones or headgear, in a watch or armband, or in a leg band or leg strap.

The method 800 includes extracting one or more movement features from the movement data, each movement feature characterizing a respective aspect of a movement pattern of the user's performance that is associated with lower extremity injury risk (804). Extracting the one or more movement features can include performing a principle component analysis to reduce the dimensionality of a feature set extracted from the movement data. The movement features may characterize a degree of fatigue.

In some examples, the movement task includes a jump-landing task and extracting the movement features includes identifying one or more movement phases of the jump-landing task in the movement data and extracting the one or more movement features from the movement phases of the movement data. The movement phases of the jump-landing task can be one or more takeoff phases, flight phases, and stance phases. The user sensors can include an accelerometer on the user, and extracting the movement features from the jump-landing task can include extracting one or more of a combination of a ground contact time during the stance phase, a pseudo-impulse during the first half of the stance phase, a pseudo-impulse during the second half of the stance phase, and a peak acceleration during the stance phase. The user sensors can include a force plate and the jump-landing task can include a combination of one or more events comprising jumping and landing on the one or more force plates, and extracting movement features can include extracting the movement features to characterize timing, loading, and asymmetrical characteristics from the one or more force plates.

In some examples, the movement task includes a drop-landing task, and extracting the movement features includes identifying one or more movement phases of the drop-landing task in the movement data and extracting the movement features from the movement phases of the movement data. The movement phases of the drop-landing task can include one or more takeoff phases, flight phases, and stance phases. The user sensors can include an accelerometer on the user, and extracting the movement features from the drop-landing task can include extracting one or more of a combination of a ground contact time during the stance phase, a pseudo-impulse during the first half of the stance phase, a pseudo-impulse during the second half of the stance phase, and a peak acceleration during the stance phase. The user sensors can include a force plate and the drop-landing task can include landing on the one or more force plates, and extracting the movement features can include extracting the movement features to characterize timing, loading, and asymmetrical characteristics from the one or more force plates.

In some examples, the movement task includes a countermovement jump task, and extracting the movement features includes identifying one or more movement phases of the countermovement jump task in the movement data and extracting the movement features from the movement phases of the movement data. The movement phases of a countermovement jump task can include one or more takeoff phases, flight phases, and stance phases. The user sensors can include an accelerometer on the user, and extracting the movement features from the countermovement jump task can include extracting one or more of a combination of a ground contact time during the stance phase, a pseudo-impulse during the first half of the stance phase, a pseudo-impulse during the second half of the stance phase, and a peak acceleration during the stance phase. The user sensors can include a force plate and the countermovement jump task can include a combination of one or more events including jumping and landing on the one or more force plates, and extracting the one or more movement features can include extracting the one or more movement features to characterize timing, loading, and asymmetrical characteristics from the one or more force plates.

In some examples, the movement task includes a running, jogging, or walking task. In some examples, the movement task includes one of a jumping task; a running, jogging, or walking task; a cutting and sprinting task; a squatting task, a weight lifting task; and a medicine ball toss task. The user sensors can include a combination of one or more accelerometers and gyroscopes on the user, and extracting the movement features can include extracting the movement features to characterize timing, loading, efficiency, regularity, and asymmetrical characteristics.

The method 800 includes classifying the movement pattern into a classified risk category for lower extremity injury for the user based on the one or more movement features (806). Classifying the movement pattern can include supplying the movement features to a machine learning classifier trained using pre-classified training data for the movement task. The machine learning classifier can be a support vector machine (SVM) configured to construct a hyperplane to maximize, based on the pre-classified training data, a margin of separation between the risk categories. The method 800 can include displaying, on a display device, an indicator for the classified risk category for the user.

It is understood that various details of the presently disclosed subject matter may be changed without departing from the scope of the presently disclosed subject matter. Furthermore, the foregoing description is for the purpose of illustration only, and not for the purpose of limitation.

### REFERENCES

Each of the following references is hereby incorporated herein by reference in its entirety.
Begg, R., Kamruzzaman, J., 2005. A machine learning approach for automated recognition of movement patterns using basic, kinetic and kinematic gait data. Journal of Biomechanics 38, 401-408.
Bennetts, C. J., Owings, T. M., Erdemir, A., Botek, G., Cavanagh, P. R., 2013. Clustering and classification of regional peak plantar pressures of diabetic feet. Journal of Biomechanics 46, 19-25.
Bittencourt, N. F. N., Meeuwisse, W. H., Mendona, L. D., Nettel-Aguirre, A., Ocarino, J. M., Fonseca, S. T., 2016. Complex systems approach for sports injuries: moving from risk factor identification to injury pattern recognition—narrative review and new concept. British Journal of Sports Medicine 50, 1309-1314.
Boser, B. E., Guyon, I. M., Vapnik, V. N., 1992. A training algorithm for optimal margin classifiers, in: Proceedings of the Fifth Annual Workshop on Computational Learning Theory, pp. 144-152.
Brophy, R. H., Schmitz, L., Wright, R. W., Dunn, W. R., Parker, R. D., Andrish, J. T., McCarty, E. C., Spindler, K. P., 2012. Return to play and future ACL injury risk after ACL reconstruction in soccer athletes from the multicenter orthopaedic outcomes network (MOON) group. The American Journal of Sports Medicine 40, 2517-2522.
Chan, Y., Fong, D. T., Chung, M. M., Li, W., Liao, W., Yung, P. S., Chan, K., 2010. Identification of ankle sprain motion from common sporting activities by dorsal foot kinematics data. Journal of Biomechanics 43, 1965-1969.
Clermont, C. A., Osis, S. T., Phinyomark, A., Ferber, R., 2017. Kinematic gait patterns in competitive and recreational runners. Journal of Applied Biomechanics, 1-26.
Cohen, J., 1988. Statistical Power Analysis for the Behavioral Sciences. Lawrence Erlbaum Associates, Hillsdale.
Cortes, C., Vapnik, V., 1995. Support-vector networks. Machine Learning 20, 273-297.
Deluzio, K. J., Astephen, J. L., 2007. Biomechanical features of gait waveform data associated with knee osteoarthritis: An application of principal component analysis. Gait & Posture 25, 86-93.
Fukuchi, R. K., Eskofier, B. M., Duarte, M., Ferber, R., 2011. Support vector machines for detecting age-related changes in running kinematics. Journal of Biomechanics 44, 540-542.
Joliffe, I. T., 2002. Principal Component Analysis. 2 ed., Springer, New York.
Jonic, S., Jankovic, T., Gajic, V., Popvic, D., 1999. Three machine learning techniques for automatic determination of rules to control locomotion. IEEE Transactions on Biomedical Engineering 46, 300-310.
Kobsar, D., Osis, S. T., Hettinga, B. A., Ferber, R., 2014. Classification accuracy of a single tri-axial accelerometer for training background and experience level in runners. Journal of Biomechanics 47, 2508-2511.
Labbe, D. R., de Guise, J. A., Mezghani, N., Godbout, V., Grimard, G., Baillargeon, D., Lavigne, P., Fernandes, J., Ranger, P., Hagemeister, N., 2011. Objective grading of the pivot shift phenomenon using a support vector machine approach. Journal of Biomechanics 44, 1-5.
Lutrek, M., Kalua, B., 2009. Fall detection and activity recognition with machine learning. Informatica 33, 197-204.
Maki, B. E., 1997. Gait changes in older adults: Predictors of falls or indicators of fear? Journal of the American Geriatrics Society 45, 313-320.
Maurer, C., Federolf, P., von Tscharner, V., Stirling, L., Nigg, B. M., 2012. Discrimination of gender-, speed-, and shoe-dependent movement patterns in runners using full-body kinematics. Gait & Posture 36, 40-45.
Moksnes, H., Engebretsen, L., Eitzen, I., Risberg, M. A., 2013. Functional outcomes following a non-operative treatment algorithm for anterior cruciate ligament injuries in skeletally immature children 12 years and younger. A prospective cohort with 2 years follow-up. British Journal of Sports Medicine 47, 488-494.
Muniz, A., Liu, H., Lyons, K., Pahwa, R., Liu, W., Nobre, F., Nadal, J., 2010. Comparison among probabilistic neural network, support vector machine and logistic regression for evaluating the effect of subthalamic stimulation in Parkinson disease on ground reaction force during gait. Journal of Biomechanics 43, 720-726.
Noyes, F. R., Mooar, P. A., Matthews, D. S., Butler, D. L., 1983a. The symptomatic anterior cruciate-deficient knee. Part I: The long-term functional disability in athletically active individuals. Journal of Bone & Joint Surgery 65, 154-162.
Noyes, F. R., Mooar, P. A., Matthews, D. S., Butler, D. L., 1983b. The symptomatic anterior cruciate-deficient knee. Part II: The long-term functional disability in athletically active individuals. Journal of Bone & Joint Surgery 65, 163-174.
Onodera, A. N., Neto, W. P. G., Roveri, M. I., Oliveira, W. R., Sacco, I. C., 2017. Immediate effects of EVA midsole resilience and upper shoe structure on running biomechanics: A machine learning approach. PeerJ 5, e3026.
Padua, D. A., Boling, M. C., DiStefano, L. J., Onate, J. A., Beutler, A. I., Marshall, S. W., 2011. Reliability of the landing error scoring system-real time, a clinical assessment tool of jump-landing biomechanics. Journal of Sport Rehabilitation 20, 145-156.
Padua, D. A., DiStefano, L. J., Beutler, A. I., de la Motte, S. J., DiStefano, M. J., Marshall, S. W., 2015. The landing error scoring system as a screening tool for an anterior cruciate ligament injury-prevention program in elite-youth soccer athletes. Journal of Athletic Training 50, 589-595.
Padua, D. A., Marshall, S. W., Boling, M. C., Thigpen, C. A., Garrett, W. E., Beutler, A. I., 2009. The landing error scoring system (LESS) is a valid and reliable clinical assessment tool of jump-landing biomechanics: The JUMP-ACL study. The American Journal of Sports Medicine 37, 1996-2002.
Phinyomark, A., Osis, S., Hettinga, B. A., Ferber, R., 2015. Kinematic gait patterns in healthy runners: A hierarchical cluster analysis. Journal of Biomechanics 48, 3897-3904.
Shalev-Shwartz, S., Ben-David, S., 2014. Understanding Machine Learning: From Theory to Algorithms. Cambridge University Press, New York.
Silver, A. E., Lungren, M. P., Johnson, M. E., O'Driscoll, S. W., An, K., Hughes, R. E., 2006. Using support vector machines to optimally classify rotator cuff strength data and quantify post-operative strength in rotator cuff tear patients. Journal of Biomechanics 39, 973-979.
Wu, J., Wang, J., 2008. PCA-based SVM for automatic recognition of gait patterns. Journal of Applied Biomechanics 24, 83-87.

What is claimed is:

1. A method for assessing lower extremity movement quality, the method comprising:
receiving, by a movement evaluator comprising at least one processor, movement data from one or more user sensors during a user's performance of a movement task;
extracting, by the movement evaluator, one or more movement features from the movement data, each movement feature characterizing a respective aspect of a movement pattern of the user's performance that is associated with lower extremity injury risk; and
classifying, by the movement evaluator, the movement pattern into a classified risk category of a plurality of risk categories for lower extremity injury for the user based on the one or more movement features.

2. The method of claim 1, wherein classifying the movement pattern comprises supplying the one or more movement features to a machine learning classifier trained using pre-classified training data for the movement task.

3. The method of claim 2, wherein the machine learning classifier comprises a support vector machine (SVM) configured to construct a hyperplane to maximize, based on the pre-classified training data, a margin of separation between the risk categories.

4. The method of claim 1, wherein extracting the one or more movement features comprises performing a principle component analysis (PCA) to reduce the dimensionality of a feature set extracted from the movement data.

5. The method of claim 1, wherein receiving the movement data from the one or more user sensors comprises receiving the movement data from one or more force plates during the user's performance of the movement task.

6. The method of claim 1, wherein receiving the movement data from the one or more user sensors comprises receiving the movement data from one or more pressure sensors during the user's performance of the movement task.

7. The method of claim 1, wherein receiving the movement data from the one or more user sensors comprises receiving the movement data from one or more video cameras during the user's performance of the movement task.

8. The method of claim 1, wherein receiving the movement data from the one or more user sensors comprises receiving the movement data from one or more wearable electronic fitness monitors worn by the user during the user's performance of the movement task.

9. The method of claim 8, wherein the wearable electronic fitness monitor worn by the user comprises a combination of one or more accelerometers, gyroscopes, magnetometers, pressure sensors, GPS sensors, RFID sensors, HR monitors, VO2 monitors, and SmO2 monitors.

10. The method of claim 1, wherein the movement task comprises a jump-landing task, and wherein extracting the one or more movement features comprises identifying one or more movement phases of the jump-landing task in the movement data and extracting the one or more movement features from the movement phases of the movement data.

11. The method of claim 10, wherein the movement phases of the jump-landing task comprise one or more takeoff phases, flight phases, and stance phases.

12. The method of claim 10, wherein the one or more user sensors comprise an accelerometer on the user, and wherein extracting the one or more movement features from the jump-landing task comprises extracting one or more of a combination of a ground contact time during the stance phase, a pseudo-impulse during the first half of the stance phase, a pseudo-impulse during the second half of the stance phase, and a peak acceleration during the stance phase.

13. The method of claim 10, wherein the one or more user sensors comprise a force plate and the jump-landing task comprises a combination of one or more events comprising jumping and landing on the one or more force plates, and wherein extracting the one or more movement features comprises extracting the one or more movement features to characterize a plurality of timing, loading, and asymmetrical characteristics from the one or more force plates.

14. The method of claim 1, wherein the movement task comprises a drop-landing task, and wherein extracting the one or more movement features comprises identifying one or more movement phases of the drop-landing task in the movement data and extracting the one or more movement features from the movement phases of the movement data.

15. The method of claim 14, wherein the movement phases of the drop-landing task comprise one or more takeoff phases, flight phases, and stance phases.

16. The method of claim 14, wherein the one or more user sensors comprise an accelerometer on the user, and wherein extracting the one or more movement features from the drop-landing task comprises extracting one or more of a combination of a ground contact time during the stance phase, a pseudo-impulse during the first half of the stance phase, a pseudo-impulse during the second half of the stance phase, and a peak acceleration during the stance phase.

17. The method of claim 14, wherein the one or more user sensors comprise a force plate and the drop-landing task comprises landing on the one or more force plates, and wherein extracting the one or more movement features comprises extracting the one or more movement features to characterize a plurality of timing, loading, and asymmetrical characteristics from the one or more force plates.

18. The method of claim 1, wherein the movement task comprises a countermovement jump task, and wherein extracting the one or more movement features comprises identifying one or more movement phases of the countermovement jump task in the movement data and extracting the one or more movement features from the movement phases of the movement data.

19. The method of claim 18, wherein the movement phases of a countermovement jump task comprise one or more takeoff phases, flight phases, and stance phases.

20. The method of claim 18, wherein the one or more user sensors comprise an accelerometer on the user, and wherein extracting the one or more movement features from the countermovement jump task comprises extracting one or more of a combination of a ground contact time during the stance phase, a pseudo-impulse during the first half of the stance phase, a pseudo-impulse during the second half of the stance phase, and a peak acceleration during the stance phase.

21. The method of claim 18, wherein the one or more user sensors comprise a force plate and the countermovement jump task comprises a combination of one or more events comprising jumping and landing on the one or more force plates, and wherein extracting the one or more movement features comprises extracting the one or more movement features to characterize a plurality of timing, loading, and asymmetrical characteristics from the one or more force plates.

22. The method of claim 1, wherein the movement task comprises one of a jumping task; a running, jogging, or walking task; a cutting and sprinting task; a squatting task, a weight lifting task; and a medicine ball toss task.

23. The method of claim 1, comprising displaying, on a display device, an indicator for the classified risk category for the user.

24. A system for assessing lower extremity movement quality, the system comprising:
one or more user sensors;
at least one processor; and
a movement evaluator implemented using at least one processor and configured to perform operations comprising:
receiving movement data from the one or more user sensors during a user's performance of a movement task;
extracting one or more movement features from the movement data, each movement feature characterizing a respective aspect of a movement pattern of the user's performance that is associated with lower extremity injury risk; and
classifying the movement pattern into a classified risk category of a plurality of risk categories for lower extremity injury for the user based on the one or more movement features.

25. The system of claim 24, wherein classifying the movement pattern comprises supplying the one or more movement features to a machine learning classifier trained using pre-classified training data for the movement task.

26. The system of claim 25, wherein the machine learning classifier comprises a support vector machine (SVM) configured to construct a hyperplane to maximize, based on the pre-classified training data, a margin of separation between the risk categories.

27. The system of claim 24, wherein extracting the one or more movement features comprises performing a principle component analysis (PCA) to reduce a dimensionality of a feature set extracted from the movement data.

28. The system of claim 24, wherein receiving the movement data from the one or more user sensors comprises receiving the movement data from one or more force plates during the user's performance of the movement task.

29. The system of claim 24, wherein receiving the movement data from the one or more user sensors comprises receiving the movement data from one or more pressure sensors during the user's performance of the movement task.

30. The system of claim 24, wherein receiving the movement data from the one or more user sensors comprises receiving the movement data from one or more video cameras during the user's performance of the movement task.

31. The system of claim 24, wherein receiving the movement data from the one or more user sensors comprises receiving the movement data from one or more wearable electronic fitness monitors worn by the user during the user's performance of the movement task, wherein the one or more wearable electronic fitness monitors house the one or more user sensors or at least one processor or both.

32. The system of claim 31, wherein the wearable electronic fitness monitor worn by the user comprises a combination of one or more accelerometers, gyroscopes, magnetometers, pressure sensors, GPS sensors, RFID sensors, HR monitors, VO2 monitors, and SmO2 monitors.

33. The system of claim 24, wherein the movement task comprises a jump-landing task, and wherein extracting the one or more movement features comprises identifying one or more movement phases of the jump-landing task in the movement data and extracting the one or more movement features from the movement phases of the movement data.

34. The system of claim 33, wherein the movement phases of the jump-landing task comprise one or more takeoff phases, flight phases, and stance phases.

35. The system of claim 33, wherein the one or more user sensors comprise an accelerometer on the user, and wherein extracting the one or more movement features from the jump-landing task comprises extracting one or more of a combination of a ground contact time during the stance phase, a pseudo-impulse during the first half of the stance phase, a pseudo-impulse during the second half of the stance phase, and a peak acceleration during the stance phase.

36. The system of claim 33, wherein the one or more user sensors comprise a force plate and the jump-landing task comprises a combination of one or more events comprising jumping and landing on the one or more force plates, and wherein extracting the one or more movement features comprises extracting the one or more movement features to characterize a plurality of timing, loading, and asymmetrical characteristics from the one or more force plates.

37. The system of claim 24, wherein the movement task comprises a drop-landing task, and wherein extracting the one or more movement features comprises identifying one or more movement phases of the drop-landing task in the movement data and extracting the one or more movement features from the movement phases of the movement data.

38. The system of claim 37, wherein the movement phases of the drop-landing task comprise one or more takeoff phases, flight phases, and stance phases.

39. The system of claim 37, wherein the one or more user sensors comprise an accelerometer on the user, and wherein extracting the one or more movement features from the drop-landing task comprises extracting one or more of a combination of a ground contact time during the stance phase, a pseudo-impulse during the first half of the stance phase, a pseudo-impulse during the second half of the stance phase, and a peak acceleration during the stance phase.

40. The system of claim 37, wherein the one or more user sensors comprise a force plate and the drop-landing task comprises landing on the one or more force plates, and wherein extracting the one or more movement features comprises extracting the one or more movement features to characterize a plurality of timing, loading, and asymmetrical characteristics from the one or more force plates.

41. The system of claim 24, wherein the movement task comprises a countermovement jump task, and wherein extracting the one or more movement features comprises identifying one or more movement phases of the countermovement jump task in the movement data and extracting the one or more movement features from the movement phases of the movement data.

42. The system of claim 41, wherein the movement phases of a countermovement jump task comprise one or more takeoff phases, flight phases, and stance phases.

43. The system of claim 41, wherein the one or more user sensors comprise an accelerometer on the user, and wherein extracting the one or more movement features from the countermovement jump task comprises extracting one or more of a combination of a ground contact time during the stance phase, a pseudo-impulse during the first half of the stance phase, a pseudo-impulse during the second half of the stance phase, and a peak acceleration during the stance phase.

44. The system of claim 41, wherein the one or more user sensors comprise a force plate and the countermovement jump task comprises a combination of one or more events comprising jumping and landing on the one or more force plates, and wherein extracting the one or more movement features comprises extracting the one or more movement features to characterize a plurality of timing, loading, and asymmetrical characteristics from the one or more force plates.

45. The system of claim 24, wherein the movement task comprises one of a jumping task; a running, jogging, or walking task; a cutting and sprinting task; a squatting task, a weight lifting task; and a medicine ball toss task.

46. The system of claim 24, comprising a display device, wherein the operations comprise displaying, on a display device, an indicator for the classified risk category for the user.

47. A non-transitory computer readable medium storing executable instructions that when executed by at least one processor of a computer control the computer to perform operations comprising:
receiving movement data from one or more user sensors during a user's performance of a movement task;
extracting one or more movement features from the movement data, each movement feature characterizing a respective aspect of a movement pattern of the user's performance that is associated with lower extremity injury risk; and
classifying the movement pattern into a classified risk category of a plurality of a risk categories for lower extremity injury for the user based on the one or more movement features.
